(19) United States
(12) Patent Application Publication
(10) Pub. No.: US 2023/0295840 A1
(43) Pub. Date: Sep. 21, 2023

(54) SINGLE BATH CONVERSION OF PLANT DERIVED BIOMASS TO TEXTILE GRADE FIBERS

(71) Applicant: GENCREST PRIVATE LIMITED, Mumbai (IN)

(72) Inventors: Rashmi CHOWDHARY, Mumbai (IN); Anshika AGARWAL, Mumbai (IN); Nitin SHETYE, Mumbai (IN)

(21) Appl. No.: 18/013,913

(22) PCT Filed: Jul. 2, 2021
(86) PCT No.: PCT/IB2021/055942

§ 371 Date: Dec. 30, 2022

(30) Foreign Application Priority Data
Jul. 2, 2020 (IN) 202021028273

(51) Int. Cl. D01C 1/02; C12N 9/64

(52) U.S. Cl. CPC D01C 1/02; C12N 9/6454

(57) ABSTRACT

The current invention relates to methods and compositions for converting plant derived biomass to textile grade fibres by an enzymatic single bath method, which does not require draining, refilling or water washes after every treatment. The single bath conversion of raw natural fibres derived from plant based biomass to softer, finer textile grade fibres is an eco-friendly process which is efficient and conserves water. The textile grade fibres produced have same quality when compared to those derived from conventional multi bath process.

### FIELD OF INVENTION

The current invention relates to the field of obtaining textile grade fibres from plant derived biomass by enzymatic means using single bath method, without intermittent draining, refilling or washing steps after every treatment. The invention more specifically discloses a water-saving, eco-friendly method involving single bath application for several steps for enzymatic conversion of cellulosic biomass into high quality textile-grade fibres that are of the same quality as those derived from a conventional process involving multiple baths.

### BACKGROUND

Many countries in the world, including India, are facing water scarcity crisis and there is an urgent need to find ways to conserve and save water globally. Moreover, the demand for textile fibres is increasing constantly due to increase in world population, and the demand for fibres has almost doubled in the past 16 years or so. Along with providing food and clothing for the increasing world population, there is increasing need for conserving land, water and other natural resources.

Approximately 500 million tons (Mt) of crop residues are produced every year in India(Economic Survey, 2015-16, MNRE, 2016) and this may increase in future. The surplus crop residues, especially from rice, wheat and sugarcane, are usually burned on-farm. Burning of crop residues not only results in emission of significant quantity of air pollutants and greenhouse gases (GHGs) like CO₂, N₂O, CH₄, CO, NH₃, NOx, and SO₂, but also depletes soil nutrients and kills beneficial soil microbes resulting in high impact on soil quality. Biomass burning from forest regions and agriculture crop residues can emit substantial amounts of particulate matter and other pollutants into the atmosphere.

Besides residue from cereal crops, which is primarily burnt, there is biomass residue from 35 other food crops too, such as fruits, etc. For example, it is estimated that each hectare of banana plantation produces nearly 220 tons of biomass waste. These banana wastes are disposed of by the cultivators into the rivers, lakes, or dumped in low-lying areas; and causes a serious threat to biosphere due to the release of greenhouse gases (1). Data suggest that India alone produces 67383 kilotons/year of Banana pseudostem and 35397 KT/year of cotton stalk as waste (2). Efforts are being made to convert this waste biomass into value added product which will not prevent environmental pollution but also provide additional source of income to farmers. One of the possible uses is production of textile grade fibres from the fruit and vegetable waste, but the production of high-quality textile grade fibres from such waste, has not been achieved till now, especially without causing more harm to the environment by using harsh chemicals and excess of water. Even though naturally occurring fibres have several advantages such as high availability, low toxicity, and renewability, they present many challenges to produce high quality textile grade fibres that can be used to make yarns and subsequently woven to make fine quality fabrics. Many times, natural fibres like banana, jute or hemp fibres are used to make non-woven fabrics, because of difficulties with spinning them into yarns. Also, approaches used till now to make spinnable fibres from these raw natural fibres mostly use alkaline treatments, or chemical retting, which are not only harmful to environment and use a lot of water, but also do not yield fine quality spinnable fibres such as derived from cotton. These yarn fibres produced from these raw natural fibres such banana, have to be woven with 100% cotton yarn to produce fabrics. Hence, there is a need to develop methods to produce high quality textile grade fibres that can be spun into yarn to be woven , from these abundantly occurring raw natural fibres derived from plant derived agricultural waste/residual biomass.

Textile industry is a highly water intensive industry and requires gallons of water in just processing and finishing of fibres. Every step in the production of fibres, especially from naturally existing raw fibres, is followed by washing step which makes the entire process dependent on usage of large quantities of water. Such multiple washing steps require large amounts of water, and there is an urgent need to reduce the water usage in the textile industry.

The textile industry uses large amounts of electricity, fuel, and water, with corresponding greenhouse gas emissions (GHGs) and contaminated effluent. The textile industry and especially textile wet-processing is one of the largest consumers of water in manufacturing and hence one of the main producers of industrial wastewater. Also, since various chemicals are used in different textile processes like pre-treatment, dyeing, printing, and finishing, the textile wastewater contains many toxic chemicals which if not treated properly before discharging to the environment, can cause serious environmental damage. In addition, in many countries, the charges for water supply and effluent discharge are increasing. Hence, for companies to save costs and remain competitive, they need to save water and address issues related to wastewater disposal (3).

The conventional multiple bath processes in textile industry involve washing steps after every treatment in the conversion of plant derived biomass into textile grade fibers. thereafter. The subsequent cycles of cleaning and fresh treatment with water results in huge wastage of both water and chemicals, which adversely impact the environment and this poses a serious issue for sustainability.

The current invention presents an eco-friendly method of enzymatically converting plant derived cellulosic biomass into high quality textile grade fibres by using single bath without intermittent draining, refilling or washing steps after every treatment. The single bath application disclosed herein for several steps of enzymatic production of textile grade fibers from raw natural fibers results in fibres which are of same quality as those derived from a process involving multiple baths.

### BRIEF DESCRIPTION OF DRAWINGS

FIG. 1 shows the flowchart showing the steps in the method of single bath processing of raw natural fibres to textile grade fibres by enzymatic treatment with water conservation.

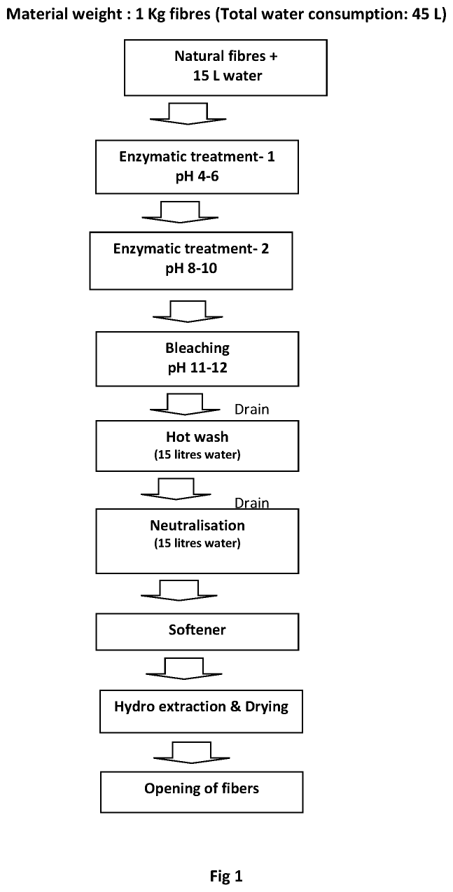

### SUMMARY

The current invention provides a method for enzymatic conversion of plant derived biomass to high grade textile fibres by a single bath process, which does not require intermittent draining, refilling and/or water washing steps in multiple baths after every treatment.

One embodiment of the current invention is a water-economical method of enzymatic conversion of raw plant fibres to textile-grade fibers, wherein the method comprises the step of performing sequential treatments of the plant derived biomass in one water bath with increasing pH milieu of the bath with every treatment, and wherein at least one of the treatments is an enzymatic treatment.

In one embodiment, at least two of the treatments are enzymatic treatments.

In one embodiment, the method further comprising the steps of:
(a) subjecting raw plant fiber bundles to a first enzymatic treatment by adding the raw fiber bundle and a first enzyme or first enzyme mixture to a water bath with acidic pH in the range of 4-6;
(b) adding a second enzyme or enzyme mixture to the same water bath from step (a) without changing the water and adjusting the pH to an alkaline pH in the range of 8-10;
(c) extracting the wet fibres, drying and opening them to obtain high quality textile grade fibres.

In one embodiment, the method above further comprises the steps of:
adding bleach to the water tank from step (b) in the method given above, after adding the second enzyme, and adjusting the pH to a more alkaline pH in the range of 11-12, to obtain bleached fibres; followed by neutralization treatment by adjusting pH of the water bath to an acidic pH in the range of 5-7, after washing the bleached fibres once with water; followed by adding softener to the water bath with pH of the water bath at pH 5-7 and temperature at 50° C. followed by extracting the wet fibres, drying and opening them to obtain high quality textile grade fibres.

In one embodiment, the method does not comprise any chemical treatment steps.

In one embodiment, the method comprises only one chemical treatment step.

In one embodiment, the only one chemical treatment step is the bleaching step after the enzymatic treatments.

In one embodiment, the method does not comprise any pre-treatment with alkali or acid.

In one embodiment, the raw plant fibre bundles are extracted from plant derived biomass by manual or machine extraction.

In one embodiment, the first enzyme or enzyme mix has pectinolytic, cellulytic, or hemicellulytic activities or a combination thereof.

In one embodiment, the second enzyme or enzyme mix has proteolytic activity.

In one embodiment, the method disclosed herein requires at least 30-50% less water than required by a multi-bath method.

In one embodiment, the method disclosed herein requires 45-50 liters of water to produce textile grade fibre from one kg of raw plant fibre. In one embodiment, the conventional multi bath process requires at least 100 liters of water to produce fibres from one kg of raw natural fibres.

In one embodiment, the water in the water bath is changed only once during the method for enzymatic plant fibre conversion to textile grade fibres in the current method.

In one embodiment, the current invention encompasses textile grade fibers produced by the method disclosed herein.

In one embodiment, the textile grade fibers produced by the method disclosed herein have a fineness of not more than 75 deniers.

In one embodiment, the textile grade fibers produced by the method disclosed herein, can be spun into yarn by automated process. In one embodiment, the yarn produced from these textile grade fibres is woven or knitted into fabrics. In one embodiment, the weaving is done by using power loom or handloom.

In one embodiment, the textile grade fibers produced by the method disclosed herein, are blended with other man-made or natural fibres.

### DETAILED DESCRIPTION

The current invention discloses methods and compositions for single bath production of textile grade fibres from plant derived biomass by enzymatic treatment. The current invention particularly relates to making high quality textile grade fibre by enzymatically processing cellulosic fibre, and using a single bath for several steps of the method of enzymatically producing textile grade fibers from cellulosic biomass, without using intermittent water washes in multiple baths at every step.

The current invention provides a method for single bath step for enzymatic conversion of plant derived biomass to high grade textile fibres without intermittent draining, refilling and/or water washing steps in multiple baths after every treatment.

### Definitions:

The term “plant derived biomass” as used herein is defined as biomass extracted from plants. It can be from plants that are specifically grown for obtaining that biomass, or can be a by-product of the main crop. Plant-derived biomass can be from any plant, including naturally growing plants, or agricultural crops.

The term “raw natural fibre” as used herein is defined as the fibers which are extracted from plant biomass using manual process or machine extractors. Examples of raw natural fibres includes, without limitation, fibres derived from Banana, hemp, Bamboo, nettle, flex, rice, ramie, jute, kenaf, sesal, abacca, coconut but not limited to these.

The term “single bath” as used herein refers to a single tank or bath or container in which all treatments steps for the conversion are done without intermittent draining, refilling and/or water washing steps. The treatment steps may be enzymatic or non-enzymatic steps. The single bath process includes the method in which all enzymatic steps along with the bleaching step are done, without intermittent draining, refilling and/or water washing steps.

The term “multiple bath” or “multiple bath process” as used herein refers to the process for converting raw natural fibers into textile grade fibers, which includes intermittent draining, refilling and/or water washing steps in between every treatment. The treatment may be enzymatic, or non-enzymatic. The multiple bath process includes the method in which all enzymatic steps along with the bleaching step are done, with intermittent draining, refilling and/or water washing steps between either every or between almost every treatment step.

“Bast fibers”are fibers that occur in the phloemorbark of certain plants. The bast fibers are in the form of bundles or strands that act as reinforcing elements and help the plant to remain erect. The main plants used for the supply of bast fibres are flax, jute, hemp, ramie and kenaf. The crude bast fibres contain less cellulose than cotton (Jute71.3%, Rawflax80.1% and decorticated ramie 83.3%). The intercellular material consists of pectins, hemi-celluloses (both complex polysaccharides) and lignin. Jute is frequently used without further purification, but flax and ramie are usually scoured and sometimes bleached.

The term “degumming” as used herein is defined as the process of separating fibers from each other by removing biomolecules such as pectins, starch, hemicellulose, gums and other biomolecules which binds the fiber together.

The terms “textile grade fibre” and “textile fibre” are used interchangeably herein, and refer to fibres that are soft and have characteristics that are suitable for making textiles. These fibres are fine and strong enough to be spun into yarns by automated process. The yarn spun from these is woven or knitted, and the weaving is done by handloom and powerloom both.

In one embodiment, the textile grade fibres produced by the single bath method disclosed herein are spinnable into yarns. In one embodiment, the textile grade fibres produced by the single bath method disclosed herein can be woven into fabrics/textiles. Tensile strength and breaking elongation are two of the most important mechanical properties for a textile grade fibre. Fibre tensile strength is often expressed by tenacity with a unit of force per denier ortex.

The term “cellulosic fibre” as used herein is defined as fibres comprising at least 20% of cellulose, and are made with ethers or esters of cellulose, which are of plant origin and can be obtained from the bark, wood or leaves of plants, or from other plant parts. In addition to cellulose, the fibres may also contain other components such pectins, hemicellulose, lignin as major components, apart from other minor constituents. With different sources percentages of these components vary altering the mechanical properties of the fibres.

The term “man-made fibres” as used herein is defined as fibres that do not exist in nature, and are usually made from various chemicals, or are regenerated from plant fibres. Examples of man-made fibres, include polyester; polyamide—(nylon); acrylics; viscose, regenerated cellulosic fibres such as rayon, bamboo fibre, Lyocell, Modal, diacetate fibre, triacetate fibre, but are not limited to these.

The term “softener” as used herein refers to textile finishing molecules, and usually softening agents applied are lubricating agents, which facilitate the fiber sliding within the fabric structure, which leads to easier deformation and creasing of the fabric. In the current invention, surfactant used are amphoteric softeners . Amphoteric softeners have good softening effects, low permanence to washing and high antistatic effects. Examples are betaine and the amine oxide type of softeners.

The term “Denier”, as used herein, is used to describe the fineness of a textile material that is quantified as the “materials' weight in grams per 9,000 meters of that material”.

The term elongation is defined as s a percentage of the starting length. The elastic elongation is of decisive importance since textile products without elasticity would hardly be usable. They must be able to deform and also return to shape. Higher elongation of textile fibres leads to easier processing and also increase comfort during wear.

Neutralization treatment is the treatment given after bleaching to neutralize the pH of the fibre bath up to 7 by adding acetic acid to the bath.

The term “breaking strength” of a fibre, as used herein, is defined as the maximum amount of tensile stress that the material can withstand before breaking or deformation. Higher breaking strength indicates higher strength of the fibre which is desirable in good quality fibres.

A loom is a device for weaving threads for getting cloth. A loom produces fabric by interlacing a series of lengthwise, parallel yarns width a series of width wise parallel yarns.

Handloom: A hand loom is a simple machine used for manual weaving.

Power loom: A power loom: is a type of loom that is powered mechanically instead of using human power to weave patterns or thread into cloth.

Weaving is a method of textile production in which two distinct sets of yarns or threads are interlaced at right angles to form a fabric or cloth. Other methods are knitting, crocheting, felting, and braiding or plaiting. The longitudinal threads are called the warp and the lateral threads are the weft, woof, or filling.

The term “non-woven fabric” as defined herein as sheet or web structures bonded together by entangling fiber or filaments (and by perforating films) mechanically, thermally, or chemically. They are flat, porous sheets that are made directly from separate fibers or from molten plastic or plastic film. They are not made by weaving or knitting and do not require converting the fibers to yarn.

A nonwoven fabric is that it is directly made from fibres or filaments without the need of converting the fibres or filaments into yarns. The production process is distinctive from traditional weaving, knitting or braiding.

The major constituent of the plant cell wall is “lignocelluloses”, which consists of lignin (15-20%), hemicellulose (25-30%) and cellulose (40-50%). These components together form a three-dimensional complex network bound by covalent and non-covalent interactions.

Lignocellulose is generally found, for example, in the fibers, pulp, stems, leaves, hulls, canes, husks, and/or cobs of plants or fibers, leaves, branches, bark, and/or wood of trees and/or bushes. Examples of lignocellulosic materials include, but are not limited to, agricultural biomass, such as farming and/or forestry material and/or residues, branches, bushes, canes, forests, grains, grasses, short rotation woody crops, herbaceous crops, and/or leaves; crop residues, such as corn, millet, and/or soybeans, herbaceous material and/or crops; forests; fruits; flowers; needles; logs; roots; saplings; shrubs; switch grasses; vegetables; fruit peels; vines; wheat midlings; oat hulls; hard and soft woods; or any combination thereof.

Hemicelluloses consist of xylan, a heteropolysaccharide substituted with monosaccharides such as L-arabinose, D-galactose, D-mannoses and organic acids such as acetic acid, ferulic acid, glucuronic acid interwoven together with help of glycosidic and ester bonds. Depolymerization of this complex polymer is essential for its efficient utilization in different industrial application.

The term “raw natural fibre” as used herein is defined as are the fibers which are mechanically extracted from plant biomass using manual process or machine extractors. Examples of raw natural fibres includes, without limitation, fibres derived from Banana, hemp, Bamboo, nettle, flex, ramie, jute, kenaf, sesal, abacca, coconut but not limited to these.

Raw fibers are derived from plant biomass by machine extractors, the machine strips the biomass open and extract the bast fibers.

The terms “enzymatic formulation”, “enzyme cocktail” and “enzymatic composition” are used interchangeably herein, and refer to a formulation comprising at least one enzyme, and it does not comprise any chemicals.

“Cellulase” or “cellulases”, as used herein, refer to an enzyme capable of hydrolyzing cellulose to glucose. Non-limiting examples of cellulases include mannan endo-1,4-β-mannosidase, 1,3-β-D-glucan glucanohydrolase, 1,3-β-glucan glucohydrolase, 1,3-1,4-β-D-glucan glucanohydrolase and 1,6-β-D-glucan glucanohydrolase.

“Xylanase” or “xylanases”, as used herein, refer to an enzyme capable of hydrolysing xylan to xylobiose and xylotriose.

Xylanase is a group of enzymes consisting of endo-1,4-β-d-xylanases (EC 3.2.1.8), β-d-xylosidases (E.C. 3.2.1.37), α-glucuronidase (EC 3.2.1.139) acetylxylan esterase (EC 3.1.1.72), α-1-arabinofuranosidases (E.C. 3.2.1.55), p-coumaric esterase (3.1.1.B10) and ferulic acid esterase (EC 3.1.1.73) involved in the depolymerization of xylan into simple monosaccharide and xylooligosaccharides.

The term “pectinase” include any acid pectinase enzyme. Pectinases are a group of enzymes that hydrolyse glycosidic linkages of pectic substances mainly poly-1,4-alpha-D-galacturonide and its derivatives which enzyme is understood to include a mature protein or a 35 precursor form thereof, or a functional fragment thereof, which essentially has the activity of the full-length enzyme. Furthermore, the term pectinase enzyme is intended to include homologues or analogues of such enzymes. Pectinases can be classified according to their preferential substrate, highly methyl-esterified pectin or low methyl-esterified pectin and polygalacturonic acid (pectate), and their reaction mechanism, beta-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by the Enzyme Nomenclature (1992) such as pectate lyase (EC 4.2.2.2), pectin lyase (EC 4.2.2.10), polygalacturonase (EC 3.2.1.15), exo-polygalacturonase (EC 3.2.1.67), exo-polygalacturonate lyase (EC 4.2.2.9) and exo-poly-alpha-galacturonosidase (EC 3.2.1.82).

Laccases, belong to the enzyme family of multi-copper oxidases (MCOs), are classified as benzenediol oxygen reductases (EC 1.10.3.2) and are also known as urushiol oxidases and p-diphenol oxidases. They are considered versatile enzymes capable of oxidizing a large number of phenolic and non-phenolic molecules due to their low substrate specificity, using oxygen as electron acceptor and generating water as a by-product.

Endo-1,4β-d-mannanase (EC 3.2.1.78) catalyzes the random cleavage of β-d-1,4-mannopyranosyl linkages within the main chain of galactomannan, glucomannan, galactoglucomannan, and mannan. They liberate short-chain β-1,4-manno-oligomers, which can be further hydrolyzed to mannose by β-mannosidases (EC 3.2.1.25).

α Amylases are starch hydrolases. Amylases are responsible for hydrolysis of starch to oligosaccharides. α-Amylase hydrolyzes the 1,4-α-glucoside bonds in compounds involving three or more molecules of glucose. β-Amylase liberates (mainly) β-maltose from starch and other compounds.

In one embodiment, the amylases used in the current invention are alpha-amylases.

As used herein, enzyme activity is defined in units. 1 unit of enzyme (U) is the amount of enzyme that catalyzes the reaction of 1 μmol of substrate per minute.

Activity definitions of enzymes used in the current invention are given below. The substrates used for activity assay can be any substrates known for the given enzymes. Moreover, the enzymes used can be from any of the known sources.

### Cellulase:

One unit of activity corresponded to the quantity of enzyme releasing 1 μmol of dextrose reducing sugar (in glucose equivalents) per minute per ml under the assay conditions. Any suitable substrate can be used for assessing the activity. The substrate used herein is Carboxymethylcellulose sodium salt-low viscosity (SIGMA-ALDRICH).

### Xylanase

One unit of activity corresponds to the quantity of enzyme releasing lumol of xylose reducing sugar (in glucose equivalents) per minute per ml under the assay conditions. The substrate used herein for assessing xylanase activity is Xylan from beechwood (SRL).

### Pectinase

One unit of activity corresponds to the quantity of enzyme releasing 1 μmol of D-galacturonic acid reducing sugar (in glucose equivalents) per minute per ml under the assay conditions. The substrate used herein is Pectin from apple (SIGMA).

### Polygalacturonase

One unit of activity corresponded to the quantity of enzyme releasing 1 μmol of D-galacturonic acid reducing sugar (in glucose equivalents) per minute per ml under the assay conditions. The substrate used for assessing the activity in the current invention is Polygalacturonic acid sodium salt (SIGMA).

### Amylase

One unit of activity corresponded to the quantity of enzyme releasing 1 μmol of maltose reducing sugar (in glucose equivalents) per minute per ml under the assay conditions. The substrate used herein for assessing the activity in the current invention is Potato starch soluble (HIMEDIA).

### Mannanase

One unit of activity corresponded to the quantity of enzyme releasing 1 μmol of mannose reducing sugar (in glucose equivalents) per minute per ml under the assay conditions. The substrate used for assessing the activity in the current invention is Locust bean gum from Ceratonia siliqua seeds (SIGMA).

Laccase: 1 IU is defined as amount of enzyme required to oxidize 1 micromole of guaiacol per min. The substrate used for assessing the activity in the current invention where the substrate is Guaiacol (SIGMA-ALDRICH).

### Embodiments

The current invention provides a method for single bath step for enzymatic conversion of plant derived biomass to high grade textile fibres by a single bath process, which does not require intermittent draining, refilling and/or water washing steps in multiple baths after every treatment.

One embodiment of the current invention is a water-economical method of enzymatic conversion of raw plant fibres to textile-grade fibers, wherein the method comprises the step of performing sequential treatments of the plant derived biomass in one water bath with increasing pH milieu of the bath with every treatment, and wherein at least one of the treatments is an enzymatic treatment.

In one embodiment, at least two of the treatments are enzymatic treatments.

In one embodiment, the method further comprising the steps of:
(c) subjecting raw plant fiber bundles to a first enzymatic treatment by adding the raw fiber bundle and a first enzyme or first enzyme mixture to a water bath with acidic pH in the range of 3-7;
(d) adding a second enzyme or enzyme mixture to the same water bath from step (a) without changing the water and adjusting the pH to an alkaline pH in the range of 8-10;
(c) extracting the wet fibres, drying and opening them to obtain high quality textile grade fibres.

In one embodiment, the method above further comprises the steps of:
adding bleach to the water tank from step (b) after adding the second enzyme, and adjusting the pH to a more alkaline pH in the range of 11-12, to obtain bleached fibres; followed by neutralization treatment by adjusting pH of the water bath to an acidic pH in the range of 5-7, after washing the bleached fibres once with water; followed by adding softener to the water bath with pH of the water bath at pH 5-7 and temperature at 50° C. followed by extracting the wet fibres, drying and opening them to obtain high quality textile grade fibres.

In one embodiment, the method does not comprise any chemical pre-treatment steps.

In one embodiment, the method comprises only one chemical treatment step, which is not a pre-treatment step.

In one embodiment, the only one chemical treatment step is the bleaching step after the enzymatic treatments.

In one embodiment, the raw plant fibre bundles are extracted from plant derived biomass by manual or machine extraction.

In one embodiment, the first enzyme or enzyme mix has pectinolytic, cellulytic, or hemicellulytic activities or a combination thereof.

In one embodiment, the second enzyme or enzyme mix has proteolytic activity.

In one embodiment, the method disclosed herein requires at least 30-50% less water than required by a multi-bath method.

In one embodiment, the method disclosed herein requires 45-50 liters of water to produce textile grade fibre from one kg of raw plant fibre. In one embodiment, the conventional multi bath process requires at least 100 liters of water to produce fibres from one kg of raw natural fibres.

In one embodiment, the water in the water bath is changed only once during the method for enzymatic plant fibre conversion to textile grade fibres in the current method.

In one embodiment, the current invention encompasses textile grade fibers produced by the method disclosed herein.

In one embodiment, the textile grade fibers produced by the method disclosed herein have a fineness of not more than 75 denier.

In one embodiment, the textile grade fibers produced by the method disclosed herein, can be spun into yarn by using power loom or handloom.

In one embodiment, the textile grade fibers produced by the method disclosed herein, wherein they are blended with other man-made or natural fibres.

One embodiment of the current invention is the method of converting plant derived biomass to textile grade fibers, the method comprising the steps of: (a) opening up the fiber bundle from the plant biomass into smaller fiber bundles by a mechanical or manual process; (b) subjecting the small fiber bundles to the first enzymatic treatment by adding the small fiber bundle and a first enzyme cocktail to a water tank; (c) second enzymatic treatment by adding the second enzyme cocktail to the water tank from the step (b); (d) adding bleach to the same water tank from steps (b) and (c) , without draining or refilling the water; (e) draining, washing with water and refilling neutralization agent in fresh water after the bleaching from step (d); and (f) softener treatment of the neutralized fibres from step (e) in the same water tank, without draining or refilling the tank, to produce textile grade fibres.

In one embodiment, raw natural fibers are derived from plant biomass by machine extractors, the machine strips the biomass open and extract the bast fibers.

In one embodiment, the first enzymatic treatment is done by pectinolytic, cellulytic, or hemicellulytic activities or a combination thereof.

In one embodiment, the first enzymatic treatment is done at an acidic pH for two hours. In one embodiment, second enzymatic treatment is done by proteolytic activities.

In one embodiment, the second enzymatic treatment is done at an alkaline pH for two hours.

In one embodiment the textile grade fiber quality produced by the method disclosed herein is same as compared to multi bath fiber production.

In one embodiment the enzymatic treatment involves addition of enzyme shaving pectino lytic, cellulytic, and/or hemicellulytic activities, or a combination thereof followed by addition of enzyme shaving proteolytic activities without performing the washing step .In one embodiment the enzymes having pectinolytic, cellulytic, or hemicellulytic activities or a combination thereof are added at acidic pH. In one embodiment the enzyme shaving pectinolytic, cellulytic, or hemicellulytic activities or a combination thereof are added for a duration of 1.5 to 2.5 hours. In one embodiment the enzymes having proteolytic activities are added at alkaline pH. In one embodiment the enzyme shaving proteolytic activities are added for a duration of 1.5 to 2.5 hours.

In one embodiment, the first enzymatic step has treatment with the enzymes Cellulase, xylanase, pectinase, polygalacturonase, lipase, alpha amylase, mannanase, laccase, and any combination thereof

In one embodiment the method as disclosed herein uses water as per material to liquor ratio. In one embodiment the material to liquor ratio (M:LR) is 1:10-1:20. In one embodiment the Material to Liquor ratio (M:LR) is 1:15.

In one embodiment, the material is the raw natural fibers, before the first enzymatic treatment. In one embodiment, they are banana or hemp fiber. In one embodiment, the material is the treated fiber obtained after the first enzymatic treatment.

In one embodiment, the liquor is water. In one embodiment, liquor is water with any other components like enzymes or mild chemicals added to it.

In one embodiment, the method further comprises mild chemical treatment post the enzymatic treatment. In one embodiment the chemical treatment is performed without the washing step.

In one embodiment, the chemical treatment is followed by removing excess water from the fibers or hydro extract. In one embodiment, the method further comprises the step of conditioning the fibers after hydro extract.

In one embodiment the method of converting raw natural fibers to textile grade fibers in a single bath comprises the step of mechanically opening the raw natural fibers.

In one embodiment, the single bath process described herein saves at least 45 liters of water for production of 1 kg of textile grade fibers from raw natural fibers.

In one embodiment the method disclosed herein uses 50% lesser water as compared to a multi bath process.

In one embodiment the method disclosed herein requires 20-30% lesser time as compared to a multi bath process.

In one embodiment, the plant derived biomass is from plants, examples of which include, but are not limited to, banana, hemp, jute, nettle, flex, bamboo, and ramie.

In one embodiment, the raw natural fibers are cellulose based fibers, wherein they comprise at least 20% of cellulose. In one embodiment, the raw natural fibers also comprise lignin, hemicellulose, pectins xylans, mannans but not limited to these.

In one embodiment, the plant derived biomass may be agricultural waste products such as banana pseudostem, hemp stalk, Flex stalk, Jute cotton stems but not limited to these.

In one embodiment, the step of enzymatically treating raw natural fibres in the method described herein may comprise treating the plant derived biomass with enzymes selected from the group consisting of pectinase, protease, cellulase, hemicellulase, amylase, and mannanase.

In one embodiment, the enzymes used for the step of enzymatic treatment of raw natural fibres are selected from the group consisting of cellulases, pectinases, hemicellulases.

In one embodiment, the method disclosed herein further comprises the step of mechanically opening the raw natural fibres before the enzymatic treatment.

In one embodiment, the method of converting the plant based raw natural fibres into textile grade fibres further comprises the step of degumming of fibers.

In one embodiment, the method disclosed herein further comprises the step of mild chemical treatment after the enzymatic treatment for enhancing whiteness index.

In one embodiment, the method disclosed herein further comprises the step of mild chemical treatment after the enzymatic treatment for enhancing whiteness index, and the mild chemical treatment is done by using alkali and/or peroxide.

One embodiment of the current invention encompasses the textile grade fibres produced by the method given herein.

In one embodiment, the textile grade fibres produced by the method disclosed herein have high tensile strength, good elongation, better spinnability, high tenacity, low resistance, finer diameter and high brightness index compared to the fibres obtained by chemical processing of raw natural fibres.

In one embodiment, the fibre diameter of the textile grade fibre produced by the method disclosed herein, is not more than 75 denier.

In one embodiment, the breaking strength of the textile grade fibre is not less than 50 g.

In one embodiment, the elongation of the fibre produced by the method described herein is not less than 5%.

In one embodiment, the brightness (degree of reflectance) is not less than 65.

In one embodiment, the textile grade fibres produced by the method disclosed herein are used for producing any material including, but not limited to, apparel, suiting-shirting, upholstery, handicrafts, doll hairs, and footwear.

In one embodiment, powerloom is used for weaving fabric from the yarn spun from the textile grade fibres produced by the method disclosed herein.

In one embodiment, handloom is used for weaving fabric from the yarn spun from the textile grade fibres produced by the method disclosed herein.

In one embodiment, the textile grade fibres produced by the method disclosed herein, are further woven or knitted.

In one embodiment, the textile grade fibres produced by the method disclosed herein are further blended with other natural or man-made fibres, examples of which include, but are not limited to, regenerated cellulosic fibres, other natural fibers, cotton, and man-made fibers.

The single bath can also be extended to include other steps of the process for converting raw natural fibers into textile grade fibers, such as, for example, eliminating the draining and refilling step between neutralization and softener steps.

### EXAMPLES

### One Bath Process

Raw banana fibres were cut to approximately 40 mm length and weigh 10 gm of banana fibres. In the stainless steel tube of launderometer (Model no. WFT8X500, JK & PC Lab Equipment, Thane), the bath was prepared by adding 5 g/1 enzyme formulation (comprising enzymes such as Cellulase 800-1000 U/ml, xylanase:10,000-15000 U/ml, pectinase:100-300 U/ml, polygalacturonase: (100-200 U/ml), lipase (500-700 U/ml), alpha amylase (300-500 U/ml), mannanase (50-100 U/ml) or laccase (10-20 U/ml), and any combination thereof, prepared in distilled water), and remaining water to keep M:LR ratio 1:15. pH of the bath was maintained to 4.0 with acetic acid.

Added 10 g of raw banana fibres in each tube, and tubes were loaded in the launderometer machine and run the machine for 120 minutes at 60° C.

Unloaded the tubes and started second enzyme treatment (alkaline protease) in the same bath by adding 1.0 g/1 protease enzyme and 0.3 g/1 sodium hydroxide to maintain pH at 9 to 10.

The machine was run for 60 minutes at temperature of 90° C.

After completion, bleaching process carried out in the same with Sodium hydroxide, Hydrogen peroxide and wetting agent at 100° C. for 60 minutes.

After bleaching drained the bath and hot wash treatment with fresh water was given at 85° C. for 10 minutes. The bath was drained again.

Neutralization treatment was done with acetic acid at 50° C. for 10 minutes.

Finally, softener treatment was given with softener at pH 5.0 and temperature 50° C. for 30 minutes. Drained the bath after treatment.

Squeezed the wet fibres and finally dry the fibres.

After drying opened the fibres manually.

The fibres were visually assessed for fineness, softness and brightness.

### Results

| | | Fineness (Fibre Denier) |
|---|---|---|
| Sr. no | Process | ASTM D1577-01/IS 234-1977 (4) |
| 1 | Raw banana fibre | 110 |
| 3 | One bath process | 68.41 |

### Conclusion

1) Enzyme treated banana fibres by one bath process showed good results for fibre fineness, softness and fibre clarity compared to any conventional multi bath process for processing raw fibres from sources such as banana, hemp etc, or from cotton seeds into high grade textile fibres.
2) One bath process saves water results in fibres with much better fineness, than raw banana fibres, and these fibres can be used in automated spinning and subsequent weaving or knitting into high grade fabrics.

### References

1. Padam et al., J Food Sci Technol (Dec. 2014) 51(12):3527-3545.
2. Naik et al Journal of Emerging Technologies and Innovative Research (Nov. 2018), Volume 5, Issue 11.
3. Hasanbeigi and Price Journal of Cleaner Production (May 2015) 95(7) Pages 30-44.
4. https://standards.globalspec.com/std/10399840/ASTM%20D1577

We claim:

2. A water-economical method of enzymatic conversion of raw plant fibres to textile-grade fibers, wherein the method comprises the step of performing sequential treatments of the plant derived biomass in one water bath with increasing pH milieu of the bath with every treatment, and wherein at least one of the treatments is an enzymatic treatment.

3. The method of claim 1, wherein at least two of the treatments are enzymatic treatments.

4. The method of claim 1, the method further comprising the steps of:
a. subjecting raw plant fiber bundles to a first enzymatic treatment by adding the raw fiber bundle and a first enzyme or first enzyme mixture to a water bath with acidic pH in the range of 3-7;
b. adding a second enzyme or enzyme mixture to the same water bath from step (a) without changing the water and adjusting the pH to an alkaline pH in the range of 8-10;
c. extracting the wet fibres, drying and opening them to obtain high quality textile grade fibres.

5. The method of claim 3, wherein the method further comprises the steps of:
a. adding bleach to the water tank from step (b) and adjusting the pH to a more alkaline pH in the range of 11-12, to obtain bleached fibres;
b. Neutralization treatment by adjusting pH of the water bath to an acidic pH in the range of 5-7, after washing the bleached fibres from step (a) once with water;
c. adding softener to the water bath with pH of the water bath at pH 5-7 and temperature at 50° C. followed by extracting the wet fibres, drying and opening them to obtain high quality textile grade fibres.

6. The method of claim 1, wherein the method does not comprise any chemical treatment steps.

7. The method of claim 1, wherein the method comprises only one chemical treatment step.

8. The method of claim 6, wherein the only one chemical treatment step is the bleaching step after the enzymatic treatments.

9. The method of claim 1, wherein the first enzyme or enzyme mix comprises cellulase, xylanase, pectinase, polygalacturonase, lipase. alpha amylase, mannanase, laccase or a combination thereof.

10. The method of claim 2, wherein the second enzyme or enzyme mix comprises at least one alkaline protease.

11. The method of claim 1, wherein it requires at least 30-50% less water than required by a multi-bath method.

12. The method of claim 1, wherein it requires 45-50 liters of water to produce textile grade fibre from one kg of raw plant fibre.

13. The method of claim 1, wherein water in the water bath is changed only once during the method for enzymatic plant fibre conversion to textile grade fibres.

14. The textile grade fibers produced by the method of claim 1.

15. The textile grade fibers of claim 13, wherein the diameter if the fibre is not more than 75 denier.
